(19) United States
(12) Patent Application Publication
(10) Pub. No.: US 2023/0321141 A1
(43) Pub. Date: Oct. 12, 2023

(54) METHOD OF USING A STYE TREATMENT FORMULATION TO TREAT AT LEAST ONE STYE, AT LEAST ONE CHALAZION, BLEPHARITIS, OR ANY COMBINATION THEREOF

(71) Applicant: Brian Brazzo, New York, NY (US)

(72) Inventor: Brian Brazzo, New York, NY (US)

(21) Appl. No.: 18/165,311

(22) Filed: Feb. 6, 2023

Related U.S. Application Data
(62) Division of application No. 17/658,255, filed on Apr. 7, 2022, now abandoned.

(51) Int. Cl. A61K 33/38; A61K 36/28; A61P 31/02

(52) U.S. Cl. CPC A61K 33/38; A61P 31/02; A61K 36/28

(57) ABSTRACT

A stye treatment formulation that includes a colloidal silver concentrate, an arnica extract, a calendula extract, niacinamide, and at least one carrier fluid is described. Methods of using the stye treatment formulation to treat at least one patient in need thereof are also described.

### FIELD

The field of the present disclosure relates to a method of using a stye treatment formulation.

### BACKGROUND

Styes are painful abscesses that form on eyelids. Styes can adversely and severely affect patients' daily lives. The discomfort of the styes can interfere with vital activities like working and sleeping. Currently, the treatments for styes include warm compresses, antibiotic ointments, and, in severe cases, local antibiotic injections (in the case of infected styes) or surgery. However, except for surgery, existing treatments do not heal the styes. Instead, the existing treatments mitigate symptoms while the styes heal independently, which does not always occur, as severe styes tend to persist. Accordingly, non-invasive treatments that can cure styes are needed.

### SUMMARY

Some embodiments of the present disclosure relate to a stye treatment formulation. In some embodiments, the stye treatment formulation may comprise, consist of, or consist essentially of a colloidal silver concentrate, an arnica extract, a calendula extract, niacinamide, and at least one carrier fluid.

Some embodiments of the present disclosure relate to a method of using the stye treatment formulation. For example, in some embodiments, the stye treatment formulation may be applied to at least one eyelid of a patient at a predetermined frequency for a predetermined duration.

Covered embodiments are defined by the claims, not the above summary. The above summary is a high-level overview of various aspects and introduces some of the further described concepts in the Detailed Description section below. The above summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

### DRAWINGS

FIGS. 1A to 3B depict eyelids of example patients before and after treatment with the formulation described herein.

### DETAILED DESCRIPTION

Some embodiments of the present disclosure relate to a stye treatment formulation. As used herein, a “stye treatment formulation” is any formulation that, when administered to an eyelid of a patient at a predetermined frequency for a predetermined duration, treats at least one of: a stye, chalazion, blepharitis, or any combination thereof. A “stye” is a pimple or abscess that forms on at least one upper eyelid, at least one lower eyelid, or any combination thereof. A “chalazion” is scar tissue that exists as a remnant of the stye. “Blepharitis” is inflammation that affects the entirety of at least one eyelid rather than being localized as a pimple or abscess. In some examples, the stye treatment formulation is administered topically to a patient. As used herein, “topically” refers to administration of the stye treatment formulation directly to a surface of the skin, such as, but not limited to a eyelid of a patient.

In some embodiments, the stye treatment formulation may comprise, consist of, or consist essentially of a colloidal silver concentrate, an arnica extract, a calendula extract, niacinamide, and at least one carrier fluid.

In some embodiments, the colloidal silver concentrate may be present in an amount ranging from: 1% to 10%, 2% to 10%, 5% to 10%, 9% to 10%, 1% to 9%, 1% to 5%, 1% to 2%, 2% to 9%, 5% to 9%, or 2% to 5% by weight of the stye treatment formulation. The colloidal silver concentrate may include colloidal silver and water in some embodiments. In some embodiments, the colloidal silver concentrate may include colloidal silver in an amount ranging from 1 ppm to 100 ppm, 25 ppm to 100 ppm, 50 ppm to 100 ppm, 75 ppm to 100 ppm, 1 ppm to 75 ppm, 1 ppm to 50 ppm, 1 ppm to 25 ppm, 25 ppm, to 75 ppm, 25 ppm to 50 ppm, or any combination thereof based on a total weight of the colloidal silver concentrate.

In certain implementations, the arnica extract may be present in an amount ranging from 1% to 10%, 2% to 10%, 5% to 10%, 9% to 10%, 1% to 9%, 1% to 5%, 1% to 2%, 2% to 9%, 5% to 9%, or 2% to 5% by weight of the stye treatment formulation. In certain examples, the arnica extract may include arnica flower, glycerin, and water.

In some aspects, the calendula extract may be present in an amount ranging from: 1% to 10%, 2% to 10%, 5% to 10%, 9% to 10%, 1% to 9%, 1% to 5%, 1% to 2%, 2% to 9%, 5% to 9%, or 2% to 5% by weight of the stye treatment formulation. In some embodiments, the calendula extract may include calendula flower, glycerin, and water.

In some aspects, the niacinamide may be present in an amount ranging from: 0.5% to 5%, 1% to 5%, 2.5% to 5%, 4% to 5%, 0.5% to 4%, 0.5% to 2.5%, 0.5% to 1%, 1% to 4%, 2.5% to 4%, or 1% to 2.5% by weight of the stye treatment formulation.

In some embodiments, the at least one carrier fluid may include water, at least one oil, at least one alcohol, or any combination thereof. In some embodiments, the at least one oil comprises coconut oil, jojoba oil, apricot oil, almond oil, olive oil, argan oil, rosehip oil, black seed oil, grape seed oil, avocado oil, sunflower oil, or any combination thereof. In certain examples, the at least one alcohol comprises ethyl alcohol, methanol, isopropyl alcohol, benzyl alcohol, or any combination thereof. In some embodiments, the at least one carrier fluid may be present in an amount ranging from: 60% to 99%, 70% to 99%, 80% to 99%, 90% to 99%, 95% to 99%, 60% to 95%, 60% to 90%, 60% to 80%, 60% to 70%, 70% to 95%, or 80% to 90% by weight of the stye treatment formulation.

In some embodiments, the stye treatment formulation may include at least one thickening agent. In some embodiments, the stye treatment formulation, where the at least one thickening agent may include tragacanth gum, guar gum, xanthan gum, or any combination thereof. In some embodiments, the at least one thickening agent may be present in an amount ranging from: 0.1% to 1%, 0.5% to 1%, 0.7% to 1%, 0.0.9% to 1%, 0.1% to 0.9%, 0.1% to 0.7%, 0.1% to 0.5%, 0.5% to 0.9%, 0.5% to 0.7%, or 0.7% to 0.9% by weight of the stye treatment formulation.

In some embodiments, the stye treatment formulation may include at least one preservative. In some embodiments, the at least one preservative may include gluconolactone, sodium benzoate, at least one paraben, phenoxyethanol, DMDM hydantoin, diazolidinyl urea, imidazolidinyl urea iodopropinyl butylcarbamate, methylisotazolinone, methylchloroiostizaolinone, benzyl alcohol, caprylic glycol, decylene glycol, ethylhexylglycerin, or any combination thereof. In some embodiments, the at least one preservative may be present in an amount ranging from: 0.1% to 1%, 0.5% to 1%, 0.7% to 1%, 0.0.9% to 1%, 0.1% to 0.9%, 0.1% to 0.7%, 0.1% to 0.5%, 0.5% to 0.9%, 0.5% to 0.7%, or 0.7% to 0.9% by weight of the stye treatment formulation.

In some embodiments, the stye treatment formulation may include at least one salt. In some embodiments, the stye treatment formulation, where the at least one salt may include sodium chloride, potassium chloride, sodium nitrate, potassium nitrate, sodium bicarbonate, calcium carbonate, calcium chloride or any combination thereof. In some embodiments, the stye treatment formulation, where the at least one salt may be present in an amount ranging from: 0.1% to 1%, 0.5% to 1%, 0.7% to 1%, 0.0.9% to 1%, 0.1% to 0.9%, 0.1% to 0.7%, 0.1% to 0.5%, 0.5% to 0.9%, 0.5% to 0.7%, or 0.7% to 0.9% by weight of the stye treatment formulation.

Some embodiments of the present disclosure relate to a method of using the stye treatment formulation described herein.

In some embodiments, the stye treatment formulation may be applied to at least one of a left upper eyelid, a left lower eyelid, a right upper eyelid, a right lower eyelid, or any combination thereof. In some embodiments, the stye treatment formulation is applied to at least one stye, at least one chalazion, blepharitis, or any combination thereof.

In some embodiments, the stye treatment formulation may be applied to at least one eyelid of a patient at a predetermined frequency for a predetermined duration. In particular examples, the predetermined frequency, the predetermined duration, or any combination thereof, may be determined by a doctor. Likewise, in specific examples, the patient may determine the predetermined frequency, the predetermined duration, or any combination thereof. In some examples, the patient is a mammal. In certain embodiments, the patient is a human.

In particular examples, the predetermined frequency may be 1 to 10 times per day, 1 to 9 times per day, 1 to 8 times per day, 1 to 7 times per day, 1 to 6 times per day, 1 to 5 times per day, 1 to 4 times per day, 1 to 3 times per day, 1 to 2 times per day, 2 to 10 times per day, 3 to 10 times per day, 4 to 10 times per day, 5 to 10 times per day, 6 to 10 times per day, 7 to 10 times per day, 8 to 10 times per day, 9 to 10 times per day, 2 to 9 times per day, 3 to 8 times per day, 4 to 7 times per day, or 5 to 6 times per day. In some examples, the predetermined frequency is at most 5 times a day, at most 4 times a day, at most 3 times a day, at most 2 times a day, or once a day.

In some implementations, the predetermined duration may be 1 to 100 weeks, 1 to 50 weeks, 5 to 50 weeks, 10 to 50 weeks, 20 to 50 weeks, 1 to 20 weeks, 1 to 10 weeks, 1 to 5 weeks, 5 to 20 weeks, 10 to 15 weeks, or 5 to 10 weeks. In some examples, the predetermined duration may be at most 100 weeks, at most 50 weeks, at most 20 weeks, at most 10 weeks, at most 5 weeks, at most 1 week, at most 5 days, at most 4 days, at most 3 days, at most 2 days, or at most 1 day.

### Example 1: Preparation

Several components were obtained. These components were mixed in a sterile vessel until fully hydrated and uniform, in the following proportions, to form the stye treatment formulation of Table 1 below. The proportions are expressed in ratios based on the weight of the carrier fluid, which in this example was water.

TABLE 1

| | Proportion (weight component |
|---|---|
| Component | per weight carrier fluid) |
| Carrier fluid (water) | 1:1 |
| Salt (sodium chloride) | 1:1000 |
| Colloidal silver concentrate (30 ppm | 7:200 |
| colloidal silver) | |
| Arnica extract | 1:30 |
| Calendula extract | 3:100 |
| Niacinamide | 17:1000 |
| Preservative (Neodefend ™ from Lotion | <1:1000 |
| Crafter ®) | |
| Thickening agent (xanthan gum) | <1:1000 |

### Example 2: Treatment

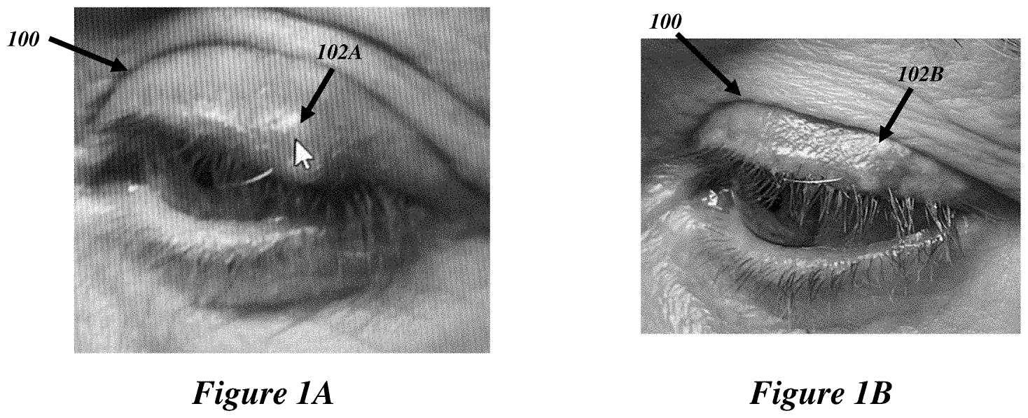

FIG. 1A depicts an untreated stye 102A on a patient's top right eyelid 100. The patient of FIG. 1A reported that the stye 102A was resistant to prior treatments. Moreover, the patient tried unsuccessfully to use prescription medication to reduce the stye 102A. The patient was directed to use the stye treatment formulation described in the present disclosure for two weeks at a predetermined frequency. The patient's treated stye 102B is shown in FIG. 1B. After treatment, the patient's stye 102B shrunk in size by approximately 50%, as estimated by the patient's doctor.

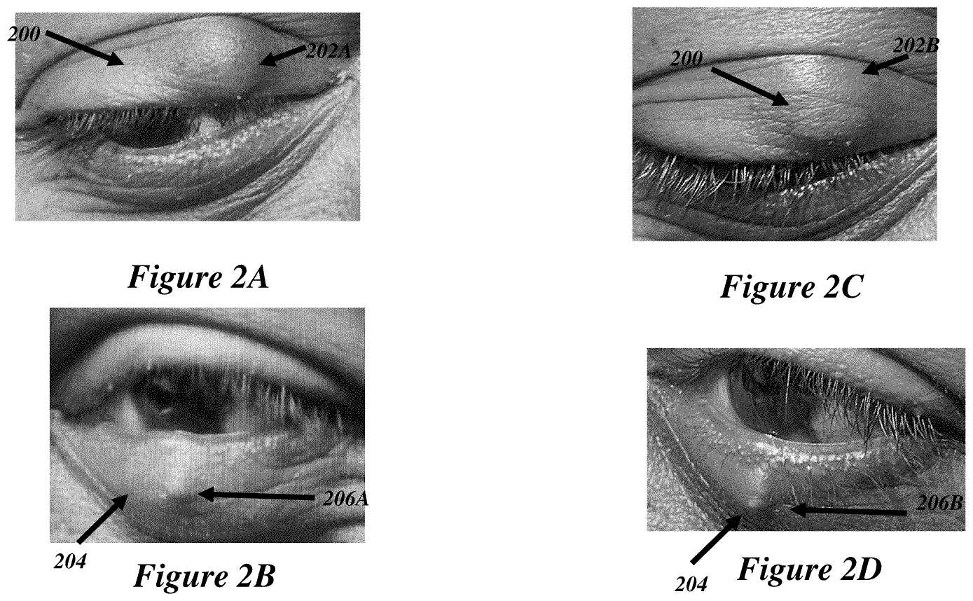

FIG. 2A depicts an untreated stye 202A on a second patient's top left eyelid 200. FIG. 2B depicts an additional untreated stye 206A on the same patient's bottom right eyelid 204. The patient of FIG. 2A tried both prescription and over-the-counter medications without success. The patient was directed to use the stye treatment formulation described in the present disclosure for a predetermined duration and a predetermined frequency. After treatment, the patient's treated styes 202B and 206B were approximately 25% smaller in size, as estimated by the patient's doctor. The results are shown in FIGS. 2C and 2D.

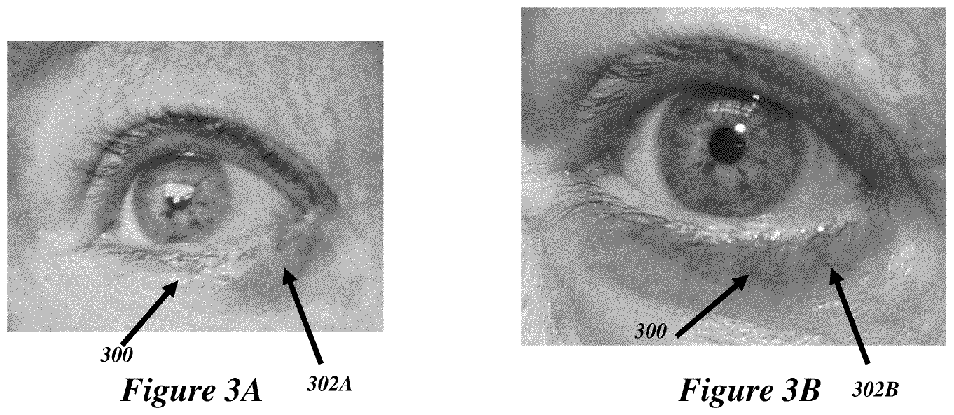

FIG. 3A depicts an untreated stye 302A on a third patient's bottom right eyelid 300. The patient was directed to use the stye treatment formulation described in the present disclosure for three weeks a predetermined frequency. The patient's treated stye 302B is shown in FIG. 3B. After treatment, the patient's treated stye 302B completely healed (i.e., shrunk by approximately 100%), as determined by the patient's doctor. The treated stye 302B consisted of only redness—no bump remained.

Table 2 below is a compilation of additional patients treated with the stye treatment formulation described herein. Comments from the patients and their doctor are provided.

TABLE 2

| | Treatment | Doctor | |
|---|---|---|---|
| Patient | duration | comments | Patient comments |
| 1 | 2 months | | |
| 2 | 1 year, | fewer styes, less | less severe chalazions |
| | variable | severe, faster | |
| | | healing | |
| 3 | 9 months | lesions resolved | no flare ups with stye treatment |
| | | | formulation |
| 4 | 1 year | fewer and less | |
| | | severe lesions | |
| 5 | 1 year, | improved with | less red and pain |
| | variable | much less | |
| | | redness | |
| 6 | 2 months, | resolved, 4 days | |
| | after high | | |
| | stress | | |
| 7 | 9 months | resolved with | got better with stye treatment |
| | | stye treatment | formulation no more redness |
| | | formulation | |
| 8 | 3 months | resolved 1 week | |
| 9 | 9 months | redness and | finally better |
| | | lesions resolved | |
| 10 | 4 months | redness and size | no recurrence |
| | | improved | |
| 11 | 9 months | feeling good, no | |
| | | recurrences | |
| 12 | 1 month | resolved | no recurrences |

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

Throughout the specification and claims, the following terms take the meanings explicitly associated herein, unless the context clearly dictates otherwise. The phrases “in one embodiment,” “in an embodiment,” and “in some embodiments” as used herein do not necessarily refer to the same embodiment(s), though it may. Furthermore, the phrases “in another embodiment” and “in some other embodiments” as used herein do not necessarily refer to a different embodiment, although it may. All embodiments of the disclosure are intended to be combinable without departing from the scope or spirit of the disclosure.

As used herein, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

Variations, modifications and alterations to embodiments of the present disclosure described above will make themselves apparent to those skilled in the art. All such variations, modifications, alterations and the like are intended to fall within the spirit and scope of the present disclosure, limited solely by the appended claims.

While several embodiments of the present disclosure have been described, it is understood that these embodiments are illustrative only, and not restrictive, and that many modifications may become apparent to those of ordinary skill in the art. For example, all dimensions discussed herein are provided as examples only, and are intended to be illustrative and not restrictive.

Any feature or element that is positively identified in this description may also be specifically excluded as a feature or element of an embodiment of the present as defined in the claims.

As used herein, the term “consisting essentially of” limits the scope of a specific claim to the specified materials or steps and those that do not materially affect the basic and novel characteristic or characteristics of the specific claim. In some embodiments, a claim reciting “consisting essentially of” may allow for the addition of any unrecited or “optional” component that does not materially affect at least one of the following: the predetermined application frequency of the stye treatment formulation or the predetermined application duration of the stye treatment formulation.

The disclosure described herein may be practiced in the absence of any element or elements, limitation or limitations, which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure.

1. A method of treating at least one stye, the method comprising:
topically administering to at least one eyelid of a patient in need thereof, a stye treatment formulation comprising:
a colloidal silver concentrate, where the colloidal silver concentrate is present in an amount ranging from 1% to 10% by weight of the stye treatment formulation;
an arnica extract, where the arnica extract is present in an amount ranging from 1% to 10% by weight of the stye treatment formulation;
a calendula extract, where the calendula extract is present in an amount ranging from 1% to 10% by weight of the stye treatment formulation;
niacinamide, where the niacinamide is present in an amount ranging from 0.5% to 5% by weight of the stye treatment formulation; and
at least one carrier fluid.

2. The method of claim 1, where the at least one carrier fluid comprises water, at least one oil, at least one alcohol, or any combination thereof.

3. The method of claim 1, where the at least one carrier fluid is present in an amount ranging from 60% to 99% by weight of the stye treatment formulation.

4. The method of claim 1, the stye treatment formulation further comprising at least one thickening agent.

5. The method of claim 1, further comprising at least one preservative.

6. The method of claim 1, the stye treatment formulation further comprising at least one salt.

7. The method of claim 1, where the colloidal silver concentrate comprises colloidal silver in an amount ranging from 1 ppm to 100 ppm based on a total weight of the colloidal silver concentrate.

8. The method of claim 1, where the colloidal silver concentrate comprises colloidal silver and water.

9. The method of claim 1, where the arnica extract comprises arnica flower, glycerin, and water.

10. The method of claim 1, where the calendula extract comprises calendula flower, glycerin, and water.

11. The method of claim 1, where the stye treatment formulation is topically administered to the at least one eyelid of the patient for a predetermined duration.

12. The method of claim 1, where the stye treatment formulation is topically administered to the at least one eyelid of the patient at a predetermined frequency.

13. The method of claim 11, where the predetermined duration is 1 to 100 weeks.

14. The method of claim 12, where the predetermined frequency is to 10 times per day.

15. The method of claim 1, where the stye treatment formulation is topically administered to multiple eyelids.

16. The method of claim 1, where the stye treatment formulation is topically administered to a top right eyelid of the patient, a bottom right eyelid of the patient, a top left eyelid of the patient, a bottom left eyelid of the patient, or any combination thereof.

17. The method of claim 1, where the patient is a human.

18. The method of claim 1, where the patient in need thereof also requires treatment for at least one of: at least one chalazion, blepharitis, or any combination thereof.

19. A method of treating at least one chalazion, the method comprising:
topically administering to at least one eyelid of a patient in need thereof, a stye treatment formulation comprising:
a colloidal silver concentrate, where the colloidal silver concentrate is present in an amount ranging from 1% to 10% by weight of the stye treatment formulation;
an arnica extract, where the arnica extract is present in an amount ranging from 1% to 10% by weight of the stye treatment formulation;
a calendula extract, where the calendula extract is present in an amount ranging from 1% to 10% by weight of the stye treatment formulation;
niacinamide, where the niacinamide is present in an amount ranging from 0.5% to 5% by weight of the stye treatment formulation; and
at least one carrier fluid.

20. A method of treating blepharitis, the method comprising:
topically administering to at least one eyelid of a patient in need thereof, a stye treatment formulation comprising:
a colloidal silver concentrate, where the colloidal silver concentrate is present in an amount ranging from 1% to 10% by weight of the stye treatment formulation;
an arnica extract, where the arnica extract is present in an amount ranging from 1% to 10% by weight of the stye treatment formulation;
a calendula extract, where the calendula extract is present in an amount ranging from 1% to 10% by weight of the stye treatment formulation;
niacinamide, where the niacinamide is present in an amount ranging from 0.5% to 5% by weight of the stye treatment formulation; and
at least one carrier fluid.
